(19) United States
(12) Patent Application Publication
(10) Pub. No.: US 2026/0123889 A1
(43) Pub. Date: May 7, 2026

(54) METHOD AND APPARATUS FOR SIGNAL COMPRESSION

(71) Applicant: SAMSUNG ELECTRONICS CO., LTD., Suwon-si (KR)

(72) Inventor: Junyeub SUH, Suwon-si (KR)

(73) Assignee: SAMSUNG ELECTRONICS CO., LTD., Suwon-si (KR)

(21) Appl. No.: 19/308,963

(22) Filed: Aug. 25, 2025

(30) Foreign Application Priority Data
Nov. 5, 2024 (KR) 10-2024-0155138

(51) Int. Cl. A61B 5/00; A61B 5/388; H03M 7/30

(52) U.S. Cl. CPC A61B 5/7232; A61B 5/388; H03M 7/30

(57) ABSTRACT

A method and apparatus for compressing a signal are provided. The method includes dividing a neural signal into a plurality of signal sections based on whether a spike signal is detected in the neural signal; determining a compression rate corresponding to each of the plurality of signal sections; and compressing a signal portion in each of the plurality of signal sections based on the compression rate corresponding to each of the plurality of signal sections.

### CROSS-REFERENCE TO RELATED APPLICATION

This application is based on and claims priority from Korean Patent Application No. 10-2024-0155138, filed on Nov. 5, 2024, in the Korean Intellectual Property Office, the entire disclosure of which is incorporated herein by reference for all purposes.

### BACKGROUND

### 1. Field

Methods and apparatuses consistent with embodiments of the disclosure relate to a method and apparatus for signal compression.

### 2. Description of Related Art

A neural signal is a biological signal that occurs when a cell transmits information electrically or chemically. The neural signal may include a spike signal, which is a short and rapid electrical signal that occurs when a cell becomes activated. Since a measured neural signal has a large data size, there may be a limitation in storing all the data. Accordingly, a method is being used in which data for only a portion of a signal section around a spike signal is stored, and a section in which a spike signal is not detected is considered noise and deleted before storing the data.

In addition, in order to analyze a postsynaptic potential (PSP) to determine the connection status between neurons, information on a signal other than a spike signal (e.g., a non-spike signal) may also be required. Thus, a method of efficiently storing the measured neural signal without destroying the neural signal may be needed.

The above description has been possessed or acquired by the inventor(s) in the course of conceiving the present disclosure and is not necessarily an art publicly known before the present application is filed.

### SUMMARY

One or more embodiments may address at least the above problems and/or disadvantages and other disadvantages not described above. Also, the embodiments are not required to overcome the disadvantages described above, and an embodiment may not overcome any of the problems described above.

According to an aspect of the disclosure, there is provided a signal compression method including: dividing a neural signal into a plurality of signal sections based on whether a spike signal is detected in the neural signal; determining a compression rate corresponding to each of the plurality of signal sections; and compressing a signal portion in each of the plurality of signal sections based on the compression rate corresponding to each of the plurality of signal sections.

The determining of the compression rate corresponding to each of the plurality of signal sections may include: determining the compression rate corresponding to each of the plurality of signal sections based on a range of signal sizes of the signal portion in each of the plurality of signal sections.

The determining of the compression rate corresponding to each of the plurality of signal sections based on the range of the signal sizes of the signal portion in each of the plurality of signal sections may include: reducing the signal sizes of the signal portion in each of the plurality of signal sections; and determining the compression rate corresponding to each of the plurality of signal sections based on a signal with a reduced signal size in each of the plurality of signal sections.

The determining of the compression rate corresponding to each of the plurality of signal sections based on the range of the signal sizes of the signal portion in each of the plurality of signal sections may include: extracting a minimum value corresponding to a minimum signal size for each of the plurality of signal sections; and reducing the signal sizes of the signal portion in each of the plurality of signal sections based on the minimum value corresponding to each of the plurality of signal sections.

The determining of the compression rate corresponding to each of the plurality of signal sections based on the signal with the reduced signal size in each of the plurality of signal sections may include: extracting a maximum value corresponding to the signal with the reduced signal size in each of the plurality of signal sections; calculating a bit number based on the maximum value; and determining the compression rate based on the bit number.

The bit number is a minimum bit number capable of expressing the maximum value.

The signal compression method may further include: storing the minimum value as an offset, for each of the plurality of signal sections; and storing the compression rate in association with a corresponding signal section, among the plurality of signal sections.

The plurality of signal sections may include: a first-type signal section, which may include the spike signal; and a second-type signal section, which does not include the spike signal.

According to another aspect of the disclosure, there is provided a non-transitory computer-readable storage medium storing instructions that, when executed by a processor, cause the processor to perform a method including dividing a neural signal into a plurality of signal sections based on whether a spike signal is detected in the neural signal; determining a compression rate corresponding to each of the plurality of signal sections; and compressing a signal portion in each of the plurality of signal sections based on the compression rate corresponding to each of the plurality of signal sections.

According to another aspect of the disclosure, there is provided an apparatus including: one or more processors; and a memory configured to store instructions, wherein the instructions, when executed by the one or more processors individually or collectively, cause the apparatus to: divide a neural signal into a plurality of signal sections based on whether a spike signal is detected in the neural signal; determine a compression rate corresponding to each of the plurality of signal sections; and compress a signal portion in each of the plurality of signal sections based on the compression rate corresponding to each of the plurality of signal sections.

The instructions, when executed by the one or more processors individually or collectively, may cause the apparatus to: determine the compression rate corresponding to each of the plurality of signal sections based on a range of signal sizes of the signal portion in each of the plurality of signal sections.

The instructions, when executed by the one or more processors individually or collectively, may cause the apparatus to: reduce the signal sizes of the signal portion in each of the plurality of signal sections; and determine the compression rate corresponding to each of the plurality of signal sections based on a signal with a reduced signal size in each of the plurality of signal sections.

The instructions, when executed by the one or more processors individually or collectively, may cause the apparatus to: extract a minimum value corresponding to a minimum signal size for each of the plurality of signal sections; and reduce the signal sizes of the signal portion in each of the plurality of signal sections based on the minimum value corresponding to each of the plurality of signal sections.

The instructions, when executed by the one or more processors individually or collectively, may cause the apparatus to: extract a maximum value corresponding to the signal with the reduced signal size in each of the plurality of signal sections; calculate a bit number based on the maximum value; and determine the compression rate based on the bit number.

The bit number is a minimum bit number capable of expressing the maximum value.

The instructions, when executed by the one or more processors individually or collectively, may cause the apparatus to: store the minimum value as an offset, for each of the plurality of signal sections; and store the compression rate in association with a corresponding signal section, among the plurality of signal sections.

The plurality of signal sections may include: a first-type signal section, which may include the spike signal; and a second-type signal section, which does not include the spike signal.

Additional aspects of embodiments will be set forth in part in the description which follows and, in part, will be apparent from the description, or may be learned by practice of the disclosure.

### BRIEF DESCRIPTION OF DRAWINGS

The above and/or other aspects will be more apparent by describing certain embodiments with reference to the accompanying drawings, in which:

FIG. 1 is a diagram illustrating an electronic device for compressing a neural signal according to an embodiment;

FIG. 2 is a diagram illustrating an example of a configuration of an apparatus according to an embodiment;

FIG. 3 is a diagram illustrating a neural signal according to an embodiment;

FIG. 4 is a diagram illustrating a signal compression method according to an embodiment;

FIG. 5 is a flowchart illustrating a signal compression method according to an embodiment;

FIG. 6 is a flowchart illustrating a signal compression method according to an embodiment; and

FIG. 7 is a block diagram illustrating an example of an electronic device according to an embodiment.

### DETAILED DESCRIPTION

The following structural or functional description of examples is provided as an example only and various alterations and modifications may be made to the examples. Thus, an actual form of implementation is not construed as limited to the examples described herein and should be understood to include all changes, equivalents, and replacements within the idea and the technical scope of the disclosure.

Although terms such as first, second, and the like are used to describe various components, the components are not limited to the terms. These terms should be used only to distinguish one component from another component. For example, a “first” component may be referred to as a “second” component, and similarly, the “second” component may also be referred to as the “first” component.

It should be noted that when one component is described as being “connected,” “coupled,” or “joined” to another component, the first component may be directly connected, coupled, or joined to the second component, or a third component may be “connected,” “coupled,” or “joined” between the first and second components.

The singular forms “a,” “an,” and “the” are intended to include the plural forms as well, unless the context clearly indicates otherwise. As used herein, each of “A or B,” “at least one of A and B,” “at least one of A or B,” “A, B, or C,” “at least one of A, B, and C,” “at least one of A, B, or C,” and the like may include any one of the items listed together in the corresponding one of the phrases, or all possible combinations thereof. It will be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical and scientific terms have the same meanings as those commonly understood by one of ordinary skill in the art to which this disclosure pertains. Terms such as those defined in commonly used dictionaries are to be interpreted as having a meaning that is consistent with their meaning in the context of the relevant art and are not to be interpreted in an idealized or overly formal sense unless expressly so defined herein.

Hereinafter, the examples are described in detail with reference to the accompanying drawings. When describing the examples with reference to the accompanying drawings, like reference numerals refer to like components and a repeated description related thereto is omitted.

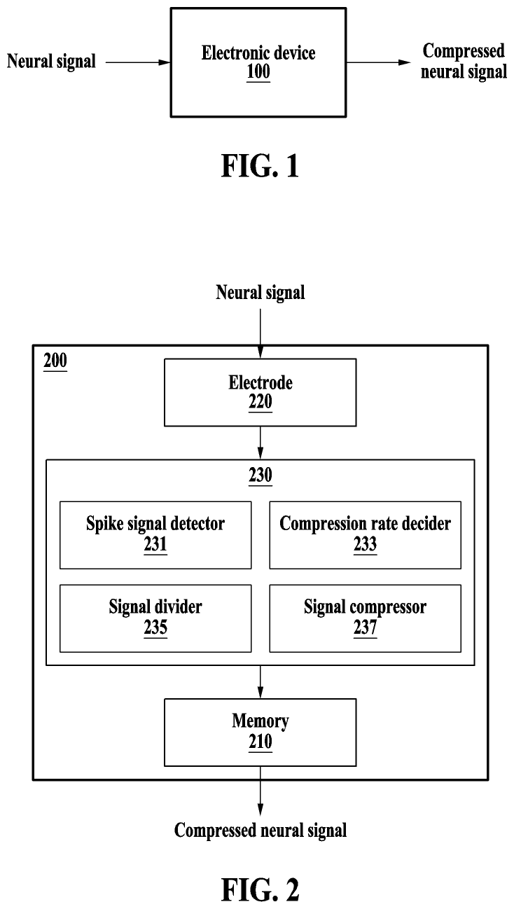

FIG. 1 is a diagram illustrating an electronic device for compressing a neural signal according to an embodiment.

Referring to FIG. 1, an electronic device 100 may receive a neural signal and may compress the neural signal to output a compressed neural signal. The neural signal may include an extracellular signal measured outside a cell and an intracellular signal measured inside the cell. The electronic device 100 may measure a neural signal of a cell (e.g., a neuron or a muscle cell). The electronic device 100 may include a microelectrode array (MEA) device. The MEA device includes a plurality of microelectrodes arranged in an array, and the MEA device may measure and record an electrical signal generated from a cell using the plurality of microelectrodes. The electronic device 100 may obtain a neural signal from a cell via a plurality of electrodes. For example, the electronic device 100 may measure a neural signal from a cell via the plurality of microelectrodes. For example, the cell may be a neuron or a muscle cell. A data size of a neural signal measured by the electronic device 100 may be determined based on a number of channels and a measurement time. In an example case in which the electronic device 100 measures 1024 channels for 1 minute at a sampling rate of 10 kHz, a size of generated data may be 10,000 (samples/sec)*2 (bytes/sample)*1024 (channels) *60(seconds)=approximately 1.228 GB (gigabytes). As a number of measurement times and/or channels increases, the data size may also increase. Thus, an efficient method of storing and managing measured data may be needed.

According to an embodiment, the electronic device 100 may detect a spike signal included in a neural signal. The spike signal is a short and rapid electrical signal that occurs when a cell becomes activated and may be called an action potential (AP). The spike signal is a basic unit by which a cell (e.g., a neuron or a muscle cell) processes and transmits information on stimuli. The spike signal may occur when a potential difference across a cell membrane changes rapidly when a cell receives a stimulus that exceeds a specific threshold value. The electronic device 100 may divide a neural signal into a plurality of signal sections based on whether a spike signal is detected within the neural signal. For example, the electronic device 100 may determine a certain section that is before and after a point at which a spike signal is detected in the neural signal as one signal section. However, the disclosure is not limited thereto, and as such, a criterion by which the electronic device 100 determines a signal section based on a spike signal may vary depending on each spike signal. For example, the electronic device 100 may determine a certain section that is before a spike signal, after the spike signal and including the spike signal as one signal section, according to a waveform of each spike signal. The electronic device 100 may determine a compression rate corresponding to each of the plurality of signal sections. The compression rate may be a rate of a data size before compression (e.g., a data size of an original neural signal) to a data size after compression (e.g., a data size of a compressed neural signal). For example, the compression rate may be expressed as a rate of a bit number used to represent the original neural signal to a bit number used to represent the compressed neural signal. The electronic device 100 may compress a signal portion included in each of the plurality of signal sections according to the compression rate. The electronic device 100 may compress the signal portion included in each signal section with a corresponding compression rate according to the compression rate corresponding to each of the plurality of signal sections. The compressed neural signal may have a data size reduced without losing information included in the neural signal. The electronic device 100 may store the compressed neural signal in a memory (e.g., a memory 210 of FIG. 2). The electronic device 100 may store an offset and the compression rate corresponding to each signal section in the memory 210 in association with the corresponding signal section. The electronic device 100 may store the offset and the compression rate in the memory 210 in a table format together with an index corresponding to each signal section. The electronic device 100 may transmit the compressed neural signal together with the stored offset and compression rate to an external device (e.g., a server or another electronic device). The compressed neural signal may be restored and used for analyzing a neural signal. For example, the compressed neural signal may be restored and used by an application. In addition to a neural signal, the electronic device 100 may measure and compress a biological signal or various signals having a specific pattern.

FIG. 2 is a diagram illustrating an example of a configuration of an apparatus according to an embodiment.

Referring to FIG. 2, an electronic device 200 (e.g., the electronic device 100 of FIG. 1) may include an electrode 220, a processor 230, and a memory 210. According to an embodiment, the processor 230 may include a spike signal detector 231, a compression rate decider 233, a signal divider 235, and a signal compressor 237. For example, the spike signal detector 231, the compression rate decider 233, the signal divider 235, and the signal compressor 237 may be software modules implemented on the processor 230 of the electronic device 200. For example, the processor 230 may execute one or more software codes or one or more instructions stored in the memory 210, and based on the execution of the one or more software code or the one or more instructions, the processor 230 may be configured to implement the spike signal detector 231, the compression rate decider 233, the signal divider 235, and the signal compressor 237. However, the disclosure is not limited thereto, and as such, one or more components of the electronic device 200 may be implemented as hardware or combination of hardware and software. For example, the spike signal detector 231 may be implemented by a spike signal detection circuit, the compression rate decider 233 may be implemented by a compression rate decider circuit, the signal divider 235 may be implemented by a signal divider circuit, and the signal compressor 237 may be implemented by a signal compressor circuit. The spike signal detector 231, the compression rate decider 233, the signal divider 235, and the signal compressor 237 are illustrated as an example to describe the electronic device 200, and embodiments may include various modifications as long as operations of the electronic device 200 described in the present disclosure may be implemented. For example, two or more components may be combined, or one or more components may be added or omitted.

The electrode 220 may receive a neural signal (e.g., a potential) of a cell (e.g., a neuron or a muscle cell) and convert the neural signal into an electrical signal. According to an embodiment, the electrode 220 is illustrated as being located inside the electronic device 200. However, the disclosure is not limited thereto. For example, an electrode that measures a neural signal of a cell may be located outside the electronic device 200. The electrode 220 may be made of metal and may be attached to an outside of a cell or inserted into an inside of the cell to measure the electrical activity of the cell. The metal may include, but is not limited to, gold or platinum. The electrode 220 may include a plurality of electrodes. For example, the electrode 220 may include an electrode array. The electrode 220 may transmit a neural signal to the processor 230.

The spike signal detector 231 may detect a spike signal from a neural signal. The spike signal detector 231 may detect the spike signal by detecting a portion of a neural signal in which a signal size changes significantly. For example, the spike signal detector 231 may detect the spike signal by detecting a signal having a signal size (or a magnitude) that exceeds a reference value. The reference value may be a threshold value. In another example, the spike signal detector 231 may detect the spike signal by detecting a signal having a rate of change that exceeds a reference rate value. The reference rate value may be a threshold rate value. For example, the spike signal detector 231 may detect the spike signal through filtering. For example, the spike signal detector 231 may detect the spike signal by detecting a signal having a waveform of a predetermined form. However, the operation of the spike signal detector 231 to detect a spike signal in a neural signal is not limited to the above examples. As such, according to another embodiment, the spike signal may be detected in another manner.

The signal divider 235 may divide the neural signal into a plurality of signal sections based on whether a spike signal is detected within the neural signal. The plurality of signal sections may include a first-type signal section (e.g., a spike section) that includes a spike signal and a second-type signal section that does not include a spike signal (e.g., a non-spike section). For example, the signal divider 235 may determine a signal section (e.g., the first-type signal section) based on a point at which a spike signal is detected in a neural signal. For example, the point at which a spike signal is detected may be a peak of the spike signal or a starting point of the spike signal in a neural signal. For example, the signal divider 235 may determine the signal section by including a determined number of signals that are before the point at which a spike signal is detected and after the point at which a spike signal is detected in addition to the point at which a spike signal is detected. For example, the signal divider 235 may determine a certain section including a spike signal as one signal section (e.g., the first-type signal section) according to a waveform of each spike signal. According to an embodiment, a criterion by which the signal divider 235 determines a signal section based on a spike signal may vary depending on each spike signal and is not limited thereto. The signal divider 235 may determine a signal section between signal sections that include a spike signal as one signal section. For example, the signal divider 235 may determine a section before a spike signal is detected as one signal section (e.g., the second-type signal section). For example, the signal divider 235 may determine signals not included in the first-type signal section, which includes a spike signal, as the second-type signal section. The first-type signal section and the second-type signal section may be classified according to whether a spike signal is included. Even in signal sections of a same type, lengths of the signal sections, ranges of sizes of included signals, and data sizes may be identical to or different from each other.

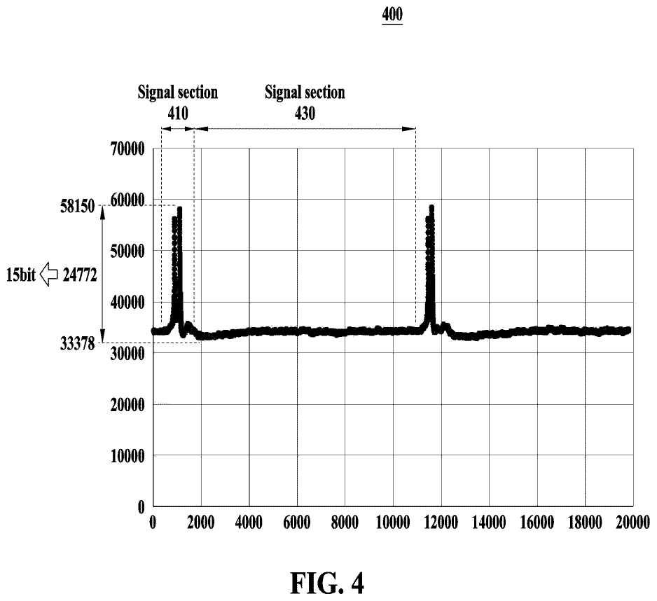

The compression rate decider 233 may determine a compression rate corresponding to each of the plurality of signal sections. The compression rate decider 233 may determine a compression rate corresponding to each of the plurality of signal sections based on a range of the signal size of a signal portion included in each of the plurality of signal sections. For example, the compression rate decider 233 may calculate a compression rate corresponding to a signal section based on a range of the signal size, which is a difference between a maximum value and a minimum value of the signal size of signals included in each signal section. The compression rate decider 233 may extract a minimum value of the signal size for each of the plurality of signal sections. The compression rate decider 233 may reduce a signal size of a signal portion included in each of the plurality of signal sections. For example, the compression rate decider 233 may redefine a signal size of each signal section by reducing the signal size of the signal portion included in each of the plurality of signal sections. The compression rate decider 233 may reduce a signal size of a signal portion of a corresponding signal section based on the minimum value extracted from each signal section. The compression rate decider 233 may determine the compression rate based on the signal with the reduced signal size. For example, the compression rate decider 233 may determine the compression rate for a signal portion with the signal size reduced and redefined according to the corresponding minimum value. The compression rate decider 233 may extract a maximum value of signal size for the signal with the signal size reduced. For example, the compression rate decider 233 may obtain a range of the signal size with the redefined signal section by extracting the maximum value of the signal size for the signal portion of each signal section, in which the signal size has been reduced. The compression rate decider 233 may calculate a bit number based on the maximum value of the signal size extracted from signals having a reduced signal size in each signal section. The compression rate decider 233 may calculate a minimum bit number that may express the maximum value of the signal size extracted from the signals having a reduced signal size in each signal section. The compression rate decider 233 may determine the compression rate corresponding to each signal section, based on a number of bits. An operation of the compression rate decider 233 to determine the compression rate corresponding to each signal section is described in detail below with reference to FIG. 4.

The signal compressor 237 may compress the signal portion included in each of the plurality of signal sections, according to the compression rate corresponding to each signal section, determined by the compression rate decider 233. The signal compressor 237 may be implemented as a circuit such as, but not limited to, a field-programmable gate array (FPGA), an integrated circuit (IC), or a printed circuit (PC). A neural signal compressed by the signal compressor 237 may be transmitted to the memory 210.

The memory 210 may store a compressed neural signal. The memory 210 may store the minimum value for each of the plurality of signal sections as an offset together with the compressed neural signal. The memory 210 may store the compression rate for each of the plurality of signal sections in association with the corresponding signal section. The compressed neural signal may be stored in the memory 210 together with the offset and the compression rate and transmitted to an external device through a communication circuit. The external device may include, but is not limited to, a server or another electronic device.

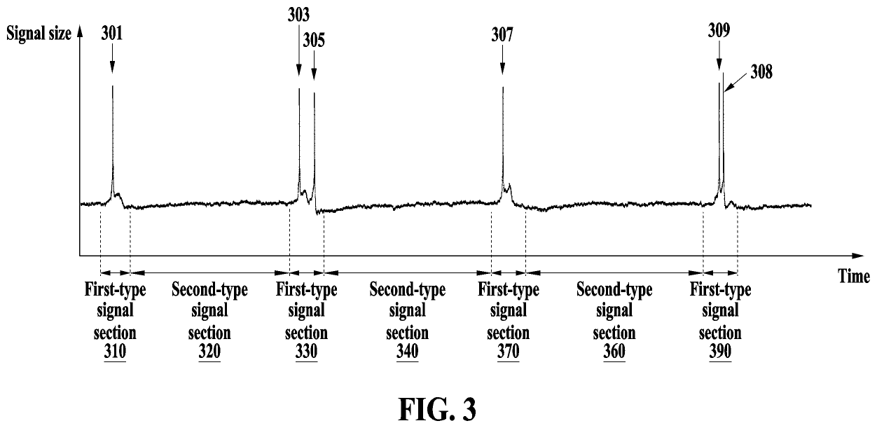

FIG. 3 is a diagram illustrating a neural signal according to an embodiment.

Referring to FIG. 3, according to an embodiment, an example of a waveform of a neural signal is illustrated. An electronic device (e.g., the electronic device 100 of FIG. 1) may receive a neural signal of a cell (e.g., a neuron or a muscle cell).

The electronic device 100 may detect a spike signal (e.g., spike signals 301, 303, 305, 307, 308 and 309) from the neural signal. For example, a spike signal detector (e.g., the spike signal detector 231 of FIG. 2) of the electronic device 100 may detect the spike signals 301, 303, 305, 307, 308 and 309 from the neural signal. A spike signal is a short and rapid electrical signal that occurs when a cell becomes activated and may be called an AP. The spike signal may occur at irregular intervals depending on an external stimulus or a state of the cell, and a frequency of the occurrence may vary.

The electronic device 100 may divide the neural signal into a plurality of signal sections based on whether a spike signal is detected within the neural signal. For example, a signal divider (e.g., the signal divider 235 of FIG. 2) of the electronic device 100 may divide the neural signal into a plurality of signal sections based on whether a spike signal is detected within the neural signal. The plurality of signal sections may include a first-type signal section (e.g., a spike section) that includes a spike signal and a second-type signal section that does not include a spike signal (e.g., a non-spike section). The first-type signal section and the second-type signal section may be classified according to whether a spike signal is included. Even in signal sections of a same type, lengths of the signal sections, ranges of sizes of included signals, and data sizes may be identical to or different from each other. For example, a plurality of first-type signal sections 310, 330, 370, and 390 may be determined as one signal section based on the spike signal 301, the spike signals 303 and 305, the spike signal 307, and the spike signals 308 and 309, respectively, and the lengths of the signal sections, the ranges of sizes of included signals, and the data sizes thereof may be identical to or different from each other. For example, the spike signals 303 and 305 may be defined one first-type signal section 330 and the spike signals 308 and 309 may be defined one first-type signal section 390.

The signal divider 235 may determine a signal section (e.g., a first-type signal section), based on a point at which a spike signal (e.g., the spike signals 301, 303, 305, 307, 308 and 309) is detected, by including a determined number of signals that are before and after the point in addition to the point. For example, the signal divider 235 may determine a section of a reference size that is before and after a point at which each of the spike signals 301, 303, 305, 307, 308 and 309 has occurred (e.g., a peak of the spike signal or a starting point of the spike signal) as one signal section. The signal divider 235 may determine a certain section including a spike signal as one signal section (e.g., the first-type signal section) according to a waveform of each spike signal. In an example case, the first-type signal section 310 may determine a certain section including the spike signal 301 as one signal section. In another example case, the first-type signal section 330 may determine a certain section including two spike signals that is determined as one signal section by considering waveforms of the spike signal 303 and the spike signal 305 adjacent to the spike signal 303.

The signal divider 235 may determine a signal section between signal sections that include a spike signal as one signal section. For example, the signal divider 235 may determine a section before a spike signal is detected as one signal section (e.g., the second-type signal section). For example, each of second-type signal sections 320, 340, and 360 may be the case of a signal section not including a spike signal determined as one signal section. A range of signal size corresponding to each signal section may be determined based on a value obtained by subtracting a minimum value of the signal size from a maximum value of the signal size, which are included in the signal section. For example, the first-type signal sections 310, 330, 370, and 390 may include a spike signal, which has abrupt changes in signal size, and may thus have a range of signal size greater than a range of signal size of a signal portion included in the second-type signal sections 320, 340, and 360.

A neural signal does not have a large change in signal size other than a spike signal, and an amplitude of a section of a negative signal size is not as high as an amplitude of a section of a positive signal size. Thus, a dynamic range of an MEA device, of which amplitudes of a negative section and a positive section are symmetrically equal, may not be effectively utilized. Therefore, a method of compressing a signal by reflecting a characteristic of each signal section may be needed.

FIG. 4 is a diagram illustrating a signal compression method according to an embodiment.

Referring to FIG. 4, an example of a neural signal received by an electronic device (e.g., the electronic device 100 of FIG. 1) is illustrated according to an embodiment. A signal size shown on the Y-axis of the graph 400 may be represented as a digital signal. For example, the Y-axis of the graph 400 may show the signal size of a neural signal measured from a cell and converted into a digital signal through an analog to digital converter (ADC).

The electronic device 100 may collect the neural signal. The electronic device 100 may divide a neural signal into a plurality of signal sections based on whether a spike signal is detected within the neural signal. For example, the electronic device 100 may determine a signal section (e.g., a signal section 410) based on a point at which a spike signal is detected (e.g., a peak of the spike signal or a starting point of the spike signal) in a neural signal, by including a number of signals that are before and after the point at which a spike signal is detected in addition to the point at which a spike signal is detected. For example, the signal section may be determined by including a predetermined number of signals that are before and after the point at which a spike signal is detected in addition to the point at which a spike signal is detected. According to an embodiment, the signal section may be determined by including signals that are within a time period before and after the point at which a spike signal is detected in addition to the point at which a spike signal is detected. For example, the electronic device 100 may determine a certain section including a spike signal as one signal section (e.g., the signal section 410) according to a waveform of each spike signal. The signal section 410 may be a first-type signal section including a spike signal. The electronic device 100 may determine a signal section between signal sections including a spike signal as one signal section. For example, the electronic device 100 may determine a signal section starting after the signal section 410 and before a next spike signal is detected as one signal section (e.g., a signal section 430). The signal section 430 may be a second-type signal section that does not include a spike signal. According to an embodiment, the signal section may be determined by including signals that are within a time period after the signal section 410 and before a next spike signal is detected.

The electronic device 100 may determine a compression rate corresponding to each of the plurality of signal sections. The electronic device 100 may extract a minimum value of the signal size for each of the plurality of signal sections. For example, the electronic device 100 may extract 33378, which is the minimum value of the signal size of a signal portion included in the signal section 410. The electronic device 100 may store the minimum value corresponding to each signal section as an offset. For example, the electronic device 100 may store 33378, which is the minimum value corresponding to the signal section 410, as an offset. In an example case in which the electronic device 100 stores the minimum value in 16 bits, a total size of stored offset data for all neural signals may be 16×W (Here, W is a number of the signal sections).

The electronic device 100 may reduce a signal size of a signal portion included in each of the plurality of signal sections. The electronic device 100 may reduce the signal size of the signal portion included in each of the plurality of signal sections, based on the extracted minimum value. The electronic device 100 may redefine the signal size of each signal section by reducing the signal size of the signal portion included in each of the plurality of signal sections. For example, the electronic device 100 may reduce the signal size by 33378, which is the minimum value of the signal size included in the signal section 410. In this case, 33378, the minimum value of the signal size of the signal section 410, may become 0.

The electronic device 100 may extract a maximum value of the signal size for a signal having a reduced signal size. For example, the electronic device 100 may extract 24772, which is the maximum value of the signal size of a signal portion having the reduced signal size, for the signal section 410. For example, as shown in FIG. 4, the electronic device 100 may extract 24772 by subtracting 33378 (which is the minimum value before the reduction of the signal size) from 58150 (which is the maximum value before the reduction of the signal size). The electronic device 100 may calculate a bit number based on the maximum value. The electronic device 100 may calculate a minimum bit number that may express the maximum value. The electronic device 100 may calculate the minimum bit number that may express the maximum value by rounding up a result of taking the log of the extracted maximum value. For example, the electronic device 100 may derive, for the signal section 410, the minimum bit number, 15 bits, by taking the log of the extracted maximum value, 24772, and rounding up the result, log 224772=14.60.

The electronic device 100 may determine the compression rate based on the number of bits. For example, the electronic device 100 may determine the compression rate corresponding to the signal section 410 based on 15 bits, the calculated number of bits. The electronic device 100 may compress a signal portion included in a signal section according to a compression rate. For example, the electronic device 100 may compress a signal portion included in each signal section with a compression rate corresponding to each of the signal section 410 and the signal section 430. The signal section 430 may be the second-type signal section that does not include a spike signal and has a very small range of signal sizes, the compression rate of the signal section 430 may be relatively high compared to the compression rate of the signal section 410. For example, the range of the signal size of the signal portion included in the signal section 430 may be approximately 154.42, and accordingly, a calculated value may be 7.28 bits, so the compression rate may be high.

The electronic device 100 may efficiently compress all data by determining the compression rate based on the range of signal size of each signal section. The electronic device 100 may have an advantage in terms of a space in which data is stored by compressing signal portions of a plurality of signal sections having various ranges of signal size by calculating a corresponding bit number instead of uniformly expressing the signal sections with a same bit number. The electronic device 100 may store the compression rate corresponding to each signal section in association with the corresponding signal section. The electronic device 100 may store an offset and the compression rate in a table format together with an index corresponding to each signal section. A size of data for the offset and the compression rate stored may be small compared to the advantage of storage space obtained by applying a different bit number to each signal section. For example, the following equation 1 illustrates a reduction in size of data by applying a different bit number to each signal section.

$$\sum {b}_{i} {w}_{i} + ( 16 \times W + 16 \times W ) \approx 0.5 ( 16 \times \sum {w}_{i} ) \qquad [ Equation 1 ]$$

Here, W denotes a total number of signal sections, bᵢ denotes a bit number corresponding to an i-th signal section, Wᵢ denotes a number of pieces of data included in the i-th signal section, 16×W denotes the size of the stored offset data (here, the offset data is assumed to be stored as 16 bits, but this may vary depending on embodiments), and 16×W denotes information on a signal included in each signal section (here, the information is assumed to be stored as 16 bits, but this may vary depending on embodiments).

Referring to [Equation 1], when adding Σbᵢwᵢ a size of data with a signal portion compressed according to the compression rate corresponding to each signal section, to 16×W+16×W, a sum of information on the compression, it may be confirmed that the result is about half of 16×W×W, a size of data expressing all signal sections in a same bit number (e.g., 16 bits). However, embodiments may vary depending on the bit number expressing the data.

Table 1 may represent, for an extracellular signal measured by the electronic 100, a result of dividing the signal into a plurality of signal sections, using a window of a predetermined size (e.g., 100 pieces of data per window), and converting data into a bit number corresponding to each signal section. The extracellular signal may be a signal measured via an electrode outside a cell membrane. The signal measured outside a cell may have a relatively small signal size compared to an intracellular signal, which may be a signal measured inside the cell membrane, and a spike signal thereof may also be attenuated and measured small.

TABLE 1

| bit | 6 | 7 | 8 | 9 | 10 | 11 | 12 |
|---|---|---|---|---|---|---|---|
| number of | 20,129 | 9,256,863 | 2,192,421 | 483,418 | 59,560 | 4,136 | 113 |
| windows | | | | | | | |

By expressing a signal that was previously stored as 16 bits in 6 bits or 12 bits, the size of the stored data may be reduced.

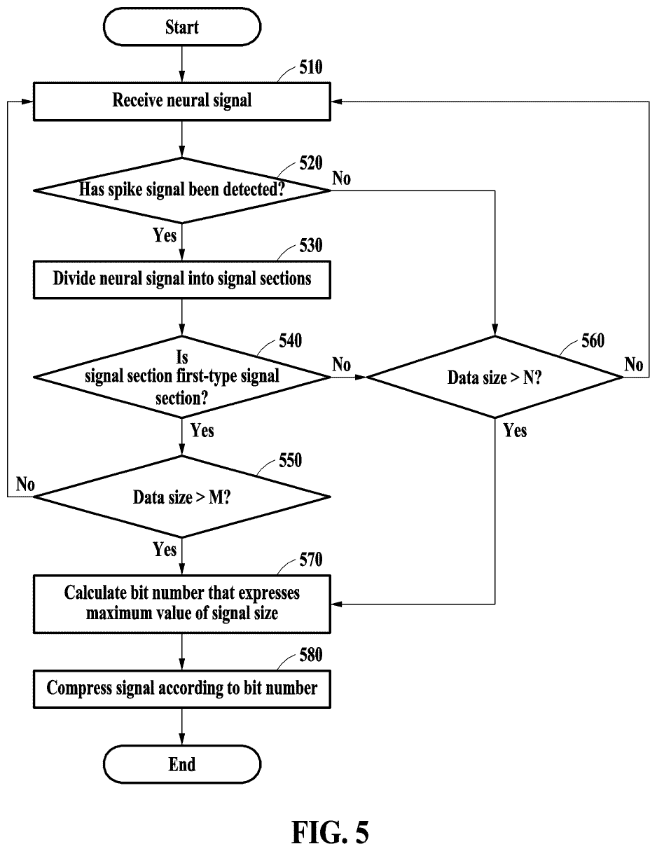

FIG. 5 is a flowchart illustrating a signal compression method according to an embodiment.

Referring to FIG. 5, according to an embodiment, operations 510 to 580 may be substantially the same as the operations of the electronic device (e.g., the electronic device 100 of FIG. 1) described with reference to FIGS. 1 to 4. Thus, a repeated description thereof is omitted.

According to an embodiment, it may be understood that operations 510 to 580 may be performed by a processor (e.g., a processor 230 illustrated in FIG. 2 or a processor 730 illustrated in FIG. 7) of the electronic device 100.

In operation 510, the method may include receiving a neural signal. For example, the electronic device 100 may receive a neural signal. The electronic device 100 may receive a neural signal of a cell via an electrode located inside and/or outside the electronic device 100.

In operation 520, the method may include determining whether a spike signal has been detected. For example, the electronic device 100 may determine whether a spike signal has been detected. The electronic device 100 may detect the spike signal by detecting a portion of a neural signal in which a signal size changes significantly. Operation 520 may be substantially the same as or similarly the operation of the spike signal detector 231 described with reference to FIG. 2, and a repeated description thereof is omitted.

According to an embodiment, based on a result of the determination in operation 520, the method may include performing operation 530 or operation 560. For example, the electronic device 100 may perform operation 530 based on detecting a spike signal. The electronic device 100 may perform operation 560 based on not detecting a spike signal.

In operation 530, the method may include dividing the neural signal into a plurality of signal sections. For example, the electronic device 100 may divide the neural signal into a plurality of signal sections. The electronic device 100 may determine a first signal section by including a determined number of signals that are before and after the spike signal, based on detecting a spike signal. The first signal section may corresponding to a first-type signal section. Among the collected neural signal data, remaining signals that are not determined to be in the same signal section as the spike signal may be determined as a second signal section. The second signal section may correspond to a second-type signal section.

In operation 540, the method may include determining whether a signal section is the first-type signal section. For example, the first type signal section may be a spike section. For example, the electronic device 100 may determine whether a signal section is the first-type signal section (e.g., a spike section). According to an embodiment, based on a result of the determination in operation 540, the method may include performing operation 550 or operation 560. For example, the electronic device 100 may perform operation 550 based on the signal section being determined as the first-type signal section. The electronic device 100 may perform operation 560 based on the signal section not being determined as the first-type signal section (e.g., being determined as the second-type signal section).

In operation 550, the method may include determining whether the size of the collected data exceeds a threshold value M (where M is a real number). For example, the electronic device 100 may determine whether the size of the collected data exceeds a threshold value M. The electronic device 100 may perform operation 550 to redefine and compress the neural signal after data of a predetermined size (e.g., M) is collected. According to an embodiment, based on a result of the determination in operation 550, the method may include performing operation 570 or operation 510. For example, the electronic device 100 may perform operation 570 based on the size of the data exceeding M or the electronic device 100 may perform operation 510 based on the size of the data not exceeding M.

In operation 560, the method may include determining whether the size of the data exceeds N (where N is a real number). For example, the electronic device 100 may determine whether the size of the data exceeds N. The electronic device 100 may perform operation 560 to redefine and compress the neural signal after data of a predetermined size (e.g., N) is collected. According to an embodiment, based on a result of the determination in operation 560, the method may include performing operation 570 or operation 510. For example, the electronic device 100 may perform operation 570 based on the size of the data exceeding N. The electronic device 100 may perform operation 510 based on the size of the data not exceeding N.

In operation 570, the method may include calculating a bit number that may express a maximum value of the signal size. For example, the electronic device 100 may calculate a bit number that may express a maximum value of the signal size. The electronic device 100 may calculate the bit number that may express the maximum value of the signal size for each signal section. Operation 570 may be substantially the same as or similar to the operation performed by the compression rate decider 233 described with reference to FIG. 2, and a repeated description thereof is omitted.

In operation 580, the method may include compressing the signal according to the bit number. For example, the electronic device 100 may compress the signal according to the bit number. The electronic device 100 may compress a signal portion included in each signal section according to a compression rate corresponding to each signal section.

According to an example, operations 510 to 580 may be performed sequentially but are not limited thereto. For example, two or more operations illustrated in FIG. 5 may be performed in parallel. According to an embodiment, the method may include one or more other operations, or omit one or more operations.

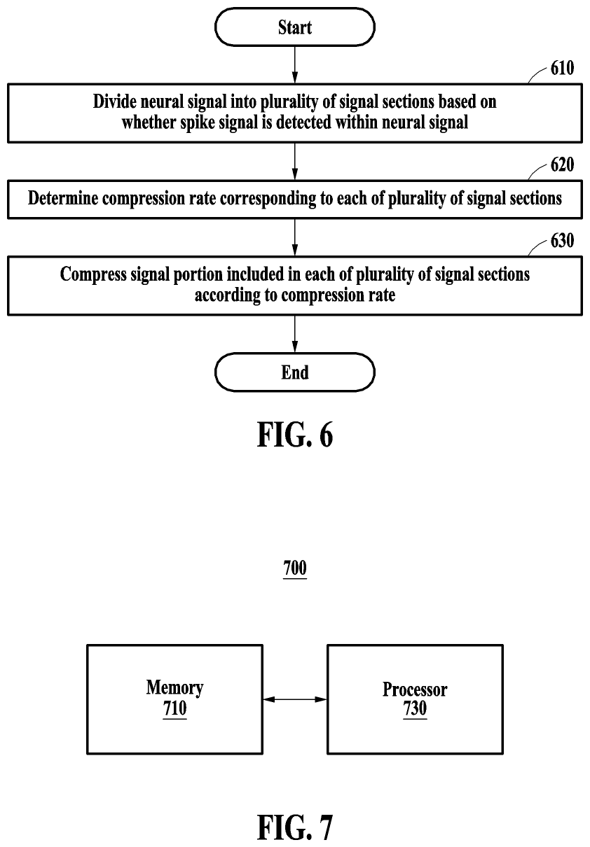

FIG. 6 is a flowchart illustrating a signal compression method according to an embodiment.

Referring to FIG. 6, operations 610, 620 and 630 may be practically same as or similar to the operations of the image segmentation apparatus (e.g., the image segmentation apparatus 100 of FIG. 1) described with reference to FIGS. 1 to 5. Thus, a repeated description thereof is omitted.

According to an embodiment, it may be understood that operations 610 to 630 may be performed by a processor (e.g., a processor 230 illustrated in FIG. 2 or a processor 730 illustrated in FIG. 7) of the electronic device 100.

In operation 610, the method may include dividing a neural signal into a plurality of neural signal sections. For example, the neural signal may be divided into the plurality of neural signal sections based on whether a spike signal is detected within the neural signal. For example, the electronic device 100 may divide a neural signal into a plurality of neural signal sections based on whether a spike signal is detected within the neural signal.

In operation 620, the method may include determining a compression rate corresponding to each of the plurality of signal sections. For example, the electronic device 100 may determine a compression rate corresponding to each of the plurality of signal sections.

In operation 630, the method may include may compressing a signal portion included in each of the plurality of signal sections according to the compression rate. For example, the electronic device 100 may compress a signal portion included in each of the plurality of signal sections according to the compression rate. The electronic device 100 may efficiently secure storage space by compressing each signal section differently according to the compression rate corresponding to each of the plurality of signal sections.

Referring to FIG. 6, according to an embodiment, operations 610 to 630 may be performed sequentially. However, embodiments are not limited thereto. For example, two or more operations may be performed in parallel. According to an embodiment, the method may include one or more other operations, or omit one or more operations.

FIG. 7 is a block diagram illustrating an example of an electronic device according to an embodiment.

Referring to FIG. 7, an electronic device 700 (e.g., the electronic device 100 of FIG. 1) may be a device that performs a neural signal compression method. The electronic device 700 may include a memory 710 and the processor 730. However, the disclosure is not limited thereto, and as such, the electronic device 700 may include one or more other components.

The memory 710 may store instructions (or programs) executable by the processor 730. For example, the instructions may include instructions for performing an operation of the processor 730 and/or an operation of each component of the processor 730.

The memory 710 may include one or more pieces of computer-readable storage media. The memory 710 may include non-volatile storage elements (e.g., a magnetic hard disk, an optical disc, a floppy disc, flash memory, erasable programmable read-only memory (EPROM), and electrically erasable programmable read-only memory (EEPROM)).

The memory 710 may be a non-transitory medium. The term “non-transitory” may indicate that the storage medium is not implemented as a carrier wave or a propagated signal. However, the term “non-transitory” should not be interpreted to mean that the memory 710 is non-movable.

The memory 710 may store instructions for an operation of dividing a neural signal into a plurality of signal sections based on whether a spike signal is detected in the neural signal, an operation of deciding a compression rate corresponding to each of the plurality of signal sections, and an operation of compressing a signal portion included in each of the plurality of signal sections according to the compression rate. However, these are only examples, and information stored in the memory 710 is not limited thereto.

The processor 730 may process data stored in the memory 710. The processor 730 may execute instructions triggered by the processor 730 and computer-readable code (e.g., software), which are stored in the memory 710.

The processor 730 may be a hardware-implemented data processing device having a circuit that is physically structured to execute desired operations. The desired operations may include, for example, codes or instructions included in a program.

For example, the hardware-implemented data processing device may include, but is not limited to, a microprocessor, a central processing unit (CPU), a processor core, a multi-core processor, a multiprocessor, an application-specific integrated circuit (ASIC), and an FPGA.

The processor 730 may cause the electronic device 700 to perform one or more operations by executing the code and/or instructions stored in the memory 710. The operations performed by the electronic device 700 may be substantially the same as the operations performed by the electronic device 100 described with reference to FIGS. 1 to 7. Thus, a repeated description thereof is omitted.

The examples described herein may be implemented using hardware components, software components, and/or combinations thereof. A processing device may be implemented using one or more general-purpose or special-purpose computers, such as, for example, a processor, a controller, an arithmetic logic unit (ALU), a digital signal processor (DSP), a microcomputer, a field-programmable gate array (FPGA), a programmable logic unit (PLU), a microprocessor or any other device capable of responding to and executing instructions in a defined manner. The processing device may run an operating system (OS) and one or more software applications that run on the OS. The processing device may also access, store, manipulate, process, and create data based on execution of the software. For purpose of simplicity, the description of a processing device is used as singular. However, one of ordinary skill in the art will appreciate that a processing device may include multiple processing elements and/or multiple types of processing elements. For example, a processing device may include a plurality of processors, or a single processor and a single controller. In addition, a different processing configuration is possible, such as one including parallel processors.

The software may include a computer program, a piece of code, an instruction, or some combination thereof, to independently or collectively instruct or configure the processing device to operate as desired. The software and/or data may be stored in any type of machine, component, physical or virtual equipment, or computer storage medium or device for the purpose of being interpreted by the processing device or providing instructions or data to the processing device. The software may also be distributed over network-coupled computer systems so that the software is stored and executed in a distributed fashion. The software and data may be stored in a non-transitory computer-readable recording medium.

The methods according to the above-described examples may be recorded in non-transitory computer-readable media including program instructions to implement various operations of the above-described examples. The media may also include the program instructions, data files, data structures, and the like alone or in combination. The program instructions recorded on the media may be those specially designed and constructed for the examples, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) and a digital versatile disc (DVD); magneto-optical media such as floptical disks; and hardware devices that are specially configured to store and perform program instructions, such as read-only memory (ROM), RAM, flash memory, and the like. Examples of program instructions include both machine code, such as those produced by a compiler, and files containing higher-level code that may be executed by the computer using an interpreter.

The above-described hardware devices may be configured to act as one or more software modules in order to perform the operations of the above-described embodiments, or vice versa.

Although the examples have been described with reference to the limited number of drawings, it will be apparent to one of ordinary skill in the art that various technical modifications and variations may be made in the examples without departing from the spirit and scope of the claims and their equivalents. For example, suitable results may be achieved if the described techniques are performed in a different order, and/or if components in a described system, architecture, device, or circuit are combined in a different manner and/or replaced or supplemented by other components or their equivalents.

Therefore, other implementations, other examples, and equivalents to the claims are also within the scope of the following claims.

What is claimed is:

1. A signal compression method comprising:
dividing a neural signal into a plurality of signal sections based on whether a spike signal is detected in the neural signal;
determining a compression rate corresponding to each of the plurality of signal sections; and
compressing a signal portion in each of the plurality of signal sections based on the compression rate corresponding to each of the plurality of signal sections.

2. The signal compression method of claim 1, wherein the determining of the compression rate corresponding to each of the plurality of signal sections comprises:
determining the compression rate corresponding to each of the plurality of signal sections based on a range of signal sizes of the signal portion in each of the plurality of signal sections.

3. The signal compression method of claim 2, wherein the determining of the compression rate corresponding to each of the plurality of signal sections based on the range of the signal sizes of the signal portion in each of the plurality of signal sections comprises:
reducing the signal sizes of the signal portion in each of the plurality of signal sections; and
determining the compression rate corresponding to each of the plurality of signal sections based on a signal with a reduced signal size in each of the plurality of signal sections.

4. The signal compression method of claim 3, wherein the determining of the compression rate corresponding to each of the plurality of signal sections based on the range of the signal sizes of the signal portion in each of the plurality of signal sections comprises:
extracting a minimum value corresponding to a minimum signal size for each of the plurality of signal sections; and
reducing the signal sizes of the signal portion in each of the plurality of signal sections based on the minimum value corresponding to each of the plurality of signal sections.

5. The signal compression method of claim 3, wherein the determining of the compression rate corresponding to each of the plurality of signal sections based on the signal with the reduced signal size in each of the plurality of signal sections comprises:
extracting a maximum value corresponding to the signal with the reduced signal size in each of the plurality of signal sections;
calculating a bit number based on the maximum value; and
determining the compression rate based on the bit number.

6. The signal compression method of claim 5, wherein the bit number is a minimum bit number capable of expressing the maximum value.

7. The signal compression method of claim 4, further comprising:
storing the minimum value as an offset, for each of the plurality of signal sections; and
storing the compression rate in association with a corresponding signal section, among the plurality of signal sections.

8. The signal compression method of claim 1, wherein the plurality of signal sections comprises:
a first-type signal section, which comprises the spike signal; and
a second-type signal section, which does not include the spike signal.

9. A non-transitory computer-readable storage medium storing instructions that, when executed by a processor, cause the processor to perform the method of claim 1.

10. An apparatus comprising:
one or more processors; and
a memory configured to store instructions,
wherein the instructions, when executed by the one or more processors individually or collectively, cause the apparatus to:
divide a neural signal into a plurality of signal sections based on whether a spike signal is detected in the neural signal;
determine a compression rate corresponding to each of the plurality of signal sections; and
compress a signal portion in each of the plurality of signal sections based on the compression rate corresponding to each of the plurality of signal sections.

11. The apparatus of claim 10, wherein the instructions, when executed by the one or more processors individually or collectively, cause the apparatus to:
determine the compression rate corresponding to each of the plurality of signal sections based on a range of signal sizes of the signal portion in each of the plurality of signal sections.

12. The apparatus of claim 11, wherein the instructions, when executed by the one or more processors individually or collectively, cause the apparatus to:
reduce the signal sizes of the signal portion in each of the plurality of signal sections; and
determine the compression rate corresponding to each of the plurality of signal sections based on a signal with a reduced signal size in each of the plurality of signal sections.

13. The apparatus of claim 12, wherein the instructions, when executed by the one or more processors individually or collectively, cause the apparatus to:
extract a minimum value corresponding to a minimum signal size for each of the plurality of signal sections; and
reduce the signal sizes of the signal portion in each of the plurality of signal sections based on the minimum value corresponding to each of the plurality of signal sections.

14. The apparatus of claim 12, wherein the instructions, when executed by the one or more processors individually or collectively, cause the apparatus to:
extract a maximum value corresponding to the signal with the reduced signal size in each of the plurality of signal sections;
calculate a bit number based on the maximum value; and
determine the compression rate based on the bit number.

15. The apparatus of claim 14, wherein the bit number is a minimum bit number capable of expressing the maximum value.

16. The apparatus of claim 13, wherein the instructions, when executed by the one or more processors individually or collectively, cause the apparatus to:
store the minimum value as an offset, for each of the plurality of signal sections; and
store the compression rate in association with a corresponding signal section, among the plurality of signal sections.

17. The apparatus of claim 10, wherein the plurality of signal sections comprises:
a first-type signal section, which comprises the spike signal; and
a second-type signal section, which does not include the spike signal.
